(19) United States
(12) Patent Application Publication
(10) Pub. No.: US 2001/0056246 A1
(43) Pub. Date: Dec. 27, 2001

(54) Visual optical device for the control of halitosis

(76) Inventors: Julio Rodriguez-Fernandez, Oviedo (ES); Regina Lopez-Fernandez, Cangas De Onis (ES); Rosario Pereiro-Garcia, Oviedo (ES); Alfredo Sanz-Medel, Gijon (ES)

Correspondence Address: STEINBERG & RASKIN, P.C., 1140 AVENUE OF THE AMERICAS, 15th FLOOR, NEW YORK, NY, 10036-5803, US

(21) Appl. No.: 09/764,825

(22) Filed: Jan. 17, 2001

(30) Foreign Application Priority Data
Jan. 18, 2000 (ES) 200000094

(51) Int. Cl. A61B 5/08; A61B 5/097; G01N 33/52

(52) U.S. Cl. 600/530; 436/120; 436/121

(57) ABSTRACT

The invention refers to a colorimetric sensor of halitosis, sensitive to volatile sulphur compounds. Said sensor comprises a solid support and a redox calorimetric reagent sensitive to volatile sulphur compounds.

The invention also refers to a device for the detection of halitosis, which comprises said sensor located inside a transparent tube. Said device undergoes a visually perceptible colour change when the concentration of volatile sulphur compounds in the analysed gas sample is higher than 200 ppb, preferably higher than 250 ppb.

### TECHNICAL FIELD OF THE INVENTION

The present invention refers to a new visual optical sensing element for the detection of volatile sulphur compounds (CVSs) in gas samples, based on a visually perceptible change of colour. This sensing element consists of a solid support on which a redox reagent sensitive to CVSs is adsorbed. Said sensor is applied mainly to the determination of volatile sulphur compounds in breath and to the indirect determination of halitosis.

Likewise, the present invention refers to the development of a visual optical device for the detection of CVSs in breath of possible halitosis patients. Said device consists of a transparent tube of an inert material in which is retained a solid sensing element according to the invention, sensitive to said sulphur compounds. Said sensing element, which consists of a solid support on which a redox reagent sensitive to the CVSs is adsorbed, undergoes a colour change upon passage thereof when the concentration of such CVSs exceeds a critical value. The device is coupled to an instrument that regulates the gas sample volume used by the device or that passes through it, to assure the homogeneity of sampling (preferably a sample of breath).

The present invention also refers to a process for halitosis detection (preferably in a patient's breath) by using the device described above.

### BACKGROUND OF THE INVENTION

Halitosis has long been associated with the presence of volatile sulphur compounds (CVSs) in breath, produced as a consequence of the degradation of food particles retained in the teeth by the bacterial flora and, in particular, by certain anaerobic bacteria existing in the periodontal bags of the oral cavity. Already in the early seventies, the production of CVSs was related to the bacterial activity. These compounds were fundamentally: hydrogen sulphide (H₂S), methylmercaptan (CH₃SH) and dimethyl sulphide, (CH₃SCH₃). Tonzetich was one of the first investigators to detect and identify these compounds by gas chromatography (J. Tonzetich, J. Periodontol., Vol. 48, (1977), 13-20). Later on, other works have ratified the findings of Tonzetich.

Halitosis is a problem of growing interest and current importance, since it affects to a great number of people (only in the United States it is considered that there are around 25 million persons suffering from chronic halitosis). In addition, an important psychological component exists in this ailment (patients that believe to have bad breath, without having it, and vice versa). However, objective methods hardly exist for its diagnosis and follow-up. Now, the most used diagnosis methods for its determination are the organoleptic qualitative tests such as, for example, the Rosemberg test (M. Rosemberg, “Bad breath. Research perspectives”, Ramot Publishing, 1995) or that of Iwakura (M. S. Alonso, I .Z. Andonegui, J. G. Hernández, ROE, Vol. 1, (1996), 97-104) that give a subjective approach to the subject problem, based on a value pattern subordinated to the smell perception capacity of the specialist who carries out the test. With the purpose of getting some less subjective results, sometimes the test is carried out with several referees, in order to eliminate the possible subjectivities inherent to the state of a certain referee in a given moment.

The search of instruments that allow objectively measuring halitosis has been directed toward the development of instruments able to quantify said volatile sulphur compounds. It is considered that CVS concentrations higher than 250 ppb are indicative of a bad breath.

The quantitative analysis of these compounds has traditionally been carried out by gas chromatography with different detectors such as, for example, a photometric flame detector (M. Rosemberg et al., J. Periodontol., Vol. 62, (1991), 487-489), or an electron capture detector (D. Clauss et al., “Bad Breath: A multidisciplinary Approach”, Ed. D. Van Steenberghe, M. Rosemberg, Leven University Press, 1996), where the volatile sulphur compounds are picked up on a column of TENAX GR., pre-concentrating them in this way for later on to be analysed by gas chromatography with electron capture detection. The great drawback of these equipments is that they are usually complex and expensive, and their operation requires highly trained personnel, so that their routine use in a physician's consulting room is to be discarded.

More recently, sensing instruments for CVSs have been developed, able to quantify bad breath in an easier way, particularly intended for use by the physician in his/her consulting room. Such instruments are usually equipments based on electrochemical measurements, as is the case of the Halimeter™ ((Interscan Corporation; U.S. Patent No. 4017373) based on electrochemical measurements by operating under controlled diffusion conditions. The gas molecules from the bad breath are adsorbed on the surface of a catalytic electrode that generates an electric current in direct ratio to the gas concentration. Later on, this electric current is converted into a voltage through a microprocessor.

Another commercial instrument used for measuring sulphur volatiles in patients suffering from halitosis is the Diamond Probe® (U.S. Pat. Nos. 5,275,161 and 5,628,312), a sulphide sensor that responds to different forms thereof (S₂, HS, H₂S and CH₃SH). It is used to measure the bacterial activity in the periodontal bags resulting from the production of CVSs due to protein degradation by anaerobic bacteria. In such a case, electric potentials are measured, whose values, in principle, are proportional to the concentration of sulphur volatiles and, therefore, to the bacterial activity.

Other patents exist (U.S. Pat. No. 4822465 and GE Patent 1510823) that disclose equipments for the determination of sulphides in gas media, but always via electrochemical measurements.

The instruments currently existent for the measurement of halitosis usually involve relatively expensive equipments based on electrochemical measurements, which seem to be suitable only for being installed in the physician's consultation.

The scope of the present invention is the development of “warning” qualitative sensors, able to evidence the presence of a threshold concentration of volatile sulphur compounds in gas samples, preferably in breath (associated to “bad odour”), in an easy way (colour change) and with a very low first cost. It is also intended that such sensors may be used by the own patient without requiring the use of more or less sophisticated measuring instruments whose purchase price and whose specialized handling rule them out of said application for the general public.

### BRIEF DESCRIPTION OF THE INVENTION

The present invention refers to a sensing element for the detection of volatile sulphur compounds (CVSs) in gaseous samples, based on a visually perceptible colour change. In a preferred embodiment of the invention, said sensor is applied to the determination of volatile sulphur compounds in breath and to the indirect determination of halitosis.

The sensing element according to the present invention comprises a solid support and a redox calorimetric reagent sensitive to CVSs.

Preferly said redox calorimetric reagent is 5,5′-dithiobis(2-nitrobenzoic acid).

As an alternative said redox calorimetric reagent is the metallic chelate Neucoproin-Cu(II).

Perferly said support is a solid support of inorganic nature.

The sensing element of the invention changes colour in a visually perceptible way as it reacts with CVSs.

On the other hand, the present invention refers to the development of a visual optical device for the detection of CVSs in gas samples, preferably in the breath of people suffering from halitosis. Said device comprises a sensing element for halitosis, according to the present invention, located inside a transparent tube to whose sides a mouthpiece of an inert material can be coupled, and an instrument to regulate the gas sample volume used by said device or that passes through it.

In a preferred embodiment of the invention, at least one sheet of a highly porous material is placed at each end of the sensing element, with the aim of retaining it, which allows the gas passing through it.

In another preferred embodiment of the present invention, between the sensing element and the porous sheet(s), at least one portion of white solid is placed in order to improve the perception of the colour change produced as a response to the presence of volatile sulphur compounds in the sample.

In a device according to the present invention, the sensing element changes colour when the concentration of CVSs in the analysed gaseous sample exceeds a certain threshold, preferably 200 ppb, more preferably 250 ppb.

Advantageously, the device according to the invention is a simple “warning” qualitative device which may be disposable and suitable for domestic uses.

The device according to the invention is applied to the detection of CVS concentrations in gas samples higher than a certain threshold of volatile sulphur compounds in gas samples. The process of the invention is characterized in that it is based in a visually perceptible colour change. Suitably, this device is applied to the detection of halitosis in the breath of a patient.

Preferly said threshold is of 200 ppb, preferably 250 ppb.

More particularly, the process comprises the following steps:

(1) passing a gas sample through an active sensing element;

(2) checking if, as a consequence of step 1, the sensing element has experienced a visually perceptible colour change.

Preferly the gas simple is breath from the bucal cavity of a patient.

### BRIEF DESCRIPTION OF THE DRAWINGS

In FIGS. 1 and 2, a sensing device for volatile sulphur compounds is shown, according to a preferred embodiment of the invention.

FIG. 1 shows the diagram of a visual optical device for the detection of halitosis according to a preferred embodiment of the present invention. Said device consists of a sensing element located inside a transparent tube (1) to whose sides a mouthpiece of an inert material (2) is coupled to facilitate to the patient the function of passing breath through the sensor, and to the other side, a bag (3) of a certain capacity that fulfils two main functions. On one hand, it controls the amount of patient's breath passing through the tube from his/her bucal cavity, so as it be the same for all measurements, while, on the other hand, it accomplishes a psychological function, the patient being able to realize how the bag is being filled, thereby making sure that he/she is insufflating breath correctly.

FIG. 2 shows a more detailed depiction of the assembly referred to as (1) in FIG. 1. The sensing element (4), which consists in a calorimetric redox reagent adsorbed on a solid support, is placed inside a transparent tube (5) of definite sizes. The transparent tube can have thick walls with the purpose of obtaining a “magnifying-glass effect” that improves the perception of the colour change occurring in the sensing element as a result of the reaction with the analytes. To both sides of the sensing element, two portions of a white solid (6) can be placed in order to improve the colour contrast and to make easier the perception of the colour change to be detected. Finally, at the ends of the tube two fragments of a highly porous material (7) can be placed with the aim of keeping firm the arrangement formed by the sensing element and the white solid inside the transparent tube.

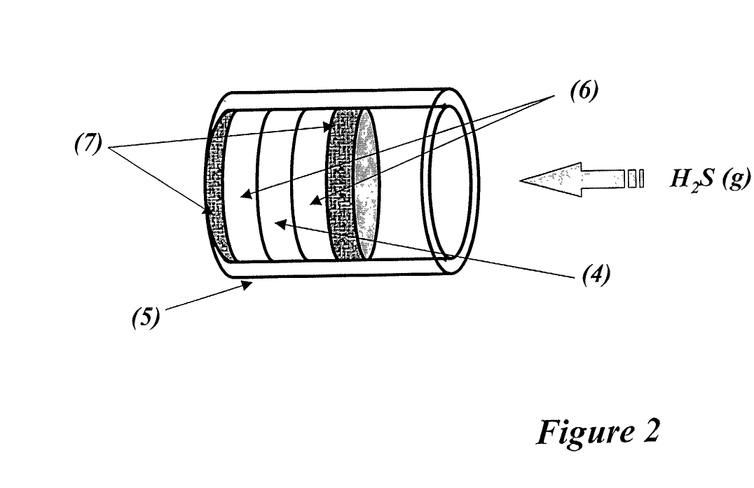

### DETAILED DESCRIPTION OF THE INVENTION

The present invention refers to the development of a sensing element for the detection of volatile sulphur compounds in gas samples, preferably in breath. Suitably, the sensing element of the invention is applied to the indirect determination of halitosis.

A sensing element according to the invention comprises a solid support and a redox calorimetric reagent sensitive to CVSs.

A redox calorimetric reagent appropriate for a sensing element according to the present invention is a reagent able of being reduced by the CVS and that has a different colour in the oxidized and reduced forms. In a sensing element according to the present invention, an appropriate quantity of redox calorimetric reagent is provided, so that the sensing element changes colour when the concentration of CVSs in the gas sample to be analysed, that has a certain volume, exceeds a certain limit.

Said calorimetric reagents should be sufficiently sensitive to volatile sulphur compounds for being employed in the development of a “warning” sensing element according to the invention, able to detect the presence of a CVS concentration higher than a certain threshold. Said threshold is preferably of 200 ppb, more preferably of 250 ppb, since concentrations higher than these values in the breath of a patient are related to halitosis or bad breath.

Appropriate calorimetric reagents according to the present invention include, for example, the 5,5′-dithiobis (2-nitrobenzoic acid) or the metallic chelate Neocuproin-Cu (II).

Suitable supports in a sensing element according to the present invention are solid supports of inorganic nature, for example silica gel.

Fixing the calorimetric reagent on the solid support can be carried out in a simple way, for example by means of a mere adsorption, since the fact of working with gas samples prevents the possibility of leaching out of the reagent from the solid support and, therefore, the requirement of a stronger interaction between reagent and solid support. This immobilization is achieved, for example, by means of a mere evaporation under reduced pressure (for example in a rotatory evaporator) of a solution of the reagent on a certain amount of solid support.

The present invention also refers to the development of a visual optical device for the detection of CVSs in gas samples. Preferably, the visual optical device of the invention is applied to the detection of CVSs in breath of people, as indirect determination of halitosis.

Said device comprises a sensing element according to the invention located inside a transparent tube. In a preferred embodiment of the invention, at an end of said transparent tube a mouthpiece of an inert material is coupled and at the other end an instrument that regulates the volume of gas sample used by said device or that passes through it.

The tube of the device according to the invention is of a transparent material, for example glass. In a preferred embodiment of the invention, the wall of the tube has a sufficient thickness to provide a magnifying effect and to improve the detectability of the colour change occurring in the sensing element as a result of the reaction with CVSs.

The mouthpiece of the device according to the invention is of an inert material, for example a plastic material. This mouthpiece has as a function to facilitate the gas sample passage through the device.

The instrument for controlling the volume of gas sample, according to the invention, has a double function. On one hand, it assures that the volume of gas sample used by the sensor or that passes through it be approximately the same in all measurements. On the other hand, in the case of halitosis detection, it has a psychological effect on the patient, since the patient becomes aware of how the bag is being filled, whereby making sure that he/she insufflates breath correctly. Said instrument can be, for example, a bag of a certain capacity.

In the design of the device of the present invention, the ratio of calorimetric reagent in the sensing element to the capacity of the instrument that regulates the sample volume is the parameter that determines the minimum concentration of analyte (CVSs) in the gas sample so that a colour change takes place visually perceptible by the human eye.

In a preferred embodiment of the invention, to each end of the sensing element is placed at least one sheet of a highly porous material, so as said sensing element be fixed inside the tube, but allowing the passage of gas through it. Said highly porous material is, for example, Nylon, Licra or some type of fabric.

In another preferred embodiment of the invention, between the sensing element and the sheet(s) of porous material is placed at least one portion of a white solid, for example silica gel, a white sheet or simply a white label, in the tube, to favour the contrast between the starting colour of the sensing element and that originated as a consequence of the reaction with the CVSs. Thus, the detection limit of the sensing device according to the invention is reduced.

Advantageously, the sensing device according to the invention allows detecting CVS concentrations higher than a certain threshold, preferably 200 ppb, more preferably 250 ppb, in gas samples. Suitably, the sensing device of the present invention can be used as a “warning” device for the detection of halitosis in a patient, without the mediation of any instrumental system.

A sensing device for halitosis according to the present invention presents a series of advantages over other currently existent sensing devices for the determination of volatile sulphur compounds in the breath. First, it is a “warning” device: only if there is a colour change (concentration of 250 ppb) the halitosis' patient should worry about his/her illness (“objective” halitosis). For this purpose, it is not necessary to use more or less expensive and complex instruments for the detection of halitosis (i.e. the “current electrochemical sensors”).

A further advantage over certain electrochemical sensing devices currently used for the detection/determination of halitosis is that it responds in the same way to hydrogen sulphide and to methylmercaptan (for example, the Halimeter™ responds much worse to methylmercaptan than to hydrogen sulphide).

Finally, the proposed sensing device is disposable and of plain use, with a very reduced purchase price, and intended for use by either the physician or the own patient, at home, so as the patient can control the state of his/her illness in a plain regular way, and without the requirement of using expensive and specialized instrumentation.

Next, some illustrative although not limitative examples of the invention are presented.

### EXAMPLES

### Example 1

### Preparation of a Sensing Element

In a 100-mL flask 50 mL of a 1.62×10⁴ M solution of 5,5′-dithiobis(2-nitrobenzoic acid), prepared by diluting 0.016 g of the acid in 250 mL methanol, and one gram silica gel, Davisil Type 646 (Aldrich, Madrid, Spain) of a particle size between 200 and 500 μm, is added. Next, the mixture is evaporated to dryness in a water bath at 25° C., in a rotatory evaporator. Once dry, the solid phase with the reagent adsorbed thereto is washed in a filtering plate of a pore size #4 with 25 mL of a 0.1 M NaHCO₃/NaOH buffer solution, pH 9.2, and then it is allowed to dry at room temperature on said plate.

The so prepared sensing element exhibits an appropriate sensibility to volatile sulphur compounds and, once being packed in a device according to the invention, it constitutes a warning sensor.

### Example 2

### Preparation of a Sensing Device

The appropriate detection of volatile sulphur compounds by using a sensing element according to the present invention (for example, the sensing element of Example 1) requires its immobilization on a solid support, as detailed in the description of the invention, to give place to a sensing device. In this example, the preparation of a sensing device is described starting from the sensing element of Example 1.

To both sides of the sensing element (4) prepared in Example 1, untreated (white) silica gel portions (6) are placed with the purpose of favouring the contrast in the colour change, whereby improving the colour perception. Since 5,5′-dithiobis(2-nitrobenzoic acid) changes colour from white to yellow, the silica gel placed to both sides of the sensing element favours the colour contrast when the 5,5′-dithiobis(2-nitrobenzoic) acid turns to yellow.

Said sensing element is introduced in a 2 cm length×3 mm inner diameter and 2,5 mm wall glass tube. Next, a mouthpiece of plastic is coupled to one of the ends of the tube, and a bag having about 300 mL capacity is coupled to the other end.

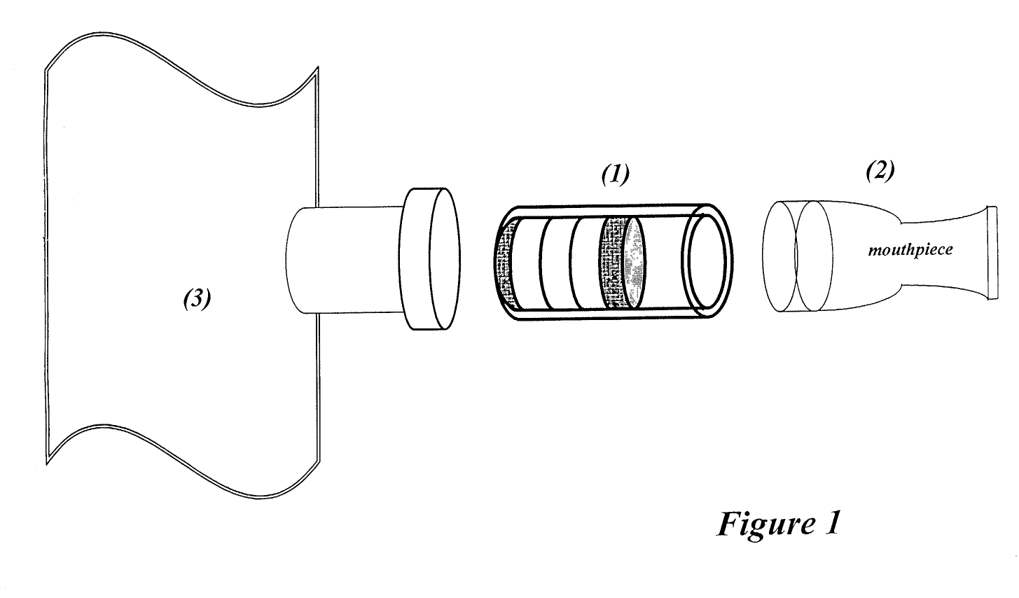

The described sensing device corresponds to the device schematised in FIG. 1.

The so developed device is sensitive to volatile sulphur compounds, and can be used for detecting the halitosis caused by said compounds (colour change when the CVS concentration exceeds 250 ppb in breath).

### Example 3

### Detection of Volatile Sulphur Compounds in Breath of Patients Suffering from Halitosis.

A series of laboratory tests and clinical trials with the sensing device described in Example 2 were carried out in order to corroborate its correct operation.

First, assays were performed with synthetic samples prepared from synthetic air to which known amounts of hydrogen sulphide or methylmercaptan had been added. In both cases, the sensor response was favourable to the required concentrations (colour change for concentrations above the limit). The colour intensity of the sensing element when reacting with the analyte increases as the analyte concentration (hydrogen sulphide, methylmercaptan) present in the gas sample increases, which may provide a certain approach to the actual concentration (low, middle or high) of the subject analyte. The fact that the sensor responds in the same way to hydrogen sulphide than to methylmercaptan offers an advantage over other types of sensors (fundamentally electrochemical) that do not respond in the same way to both analytes.

Once the assays with synthetic samples of air, with very satisfactory results, being carried out, clinical trials using volunteers. Said clinical trials with effective samples were contrasted in front of the quantitative results of a commercial equipment, like it is the Halimeter^{r}, taken in the community of periodontists like a reference instrument. The results of such comparative assays demonstrated good interrelation among the quantitative results of the Halimeter and the results of the “warning” sensor (colour change if the concentration is above 250 ppb). In the Table 1 this interrelation is demonstrated.

TABLE 1

| | |
|---|---|
| Positive Values | Negative Values |
| (>250 ppb) | (<250 ppb) |

| Halimeter ™ | Sensor | Halimeter ™ | Sensor |
|---|---|---|---|
| 491 | + | 119 | − |
| 295* | − | 119 | − |
| 1034 | + | 215 | − |
| 425 | + | 193 | − |
| 757 | + | 102 | − |
| 348 | + | 190 | − |
| 280 | + | 128 | − |
| 1658 | + | 127 | − |
| 542 | + | 156 | − |
| 408 | + | 120 | − |
| 1673 | + | 116 | − |
| 256 | + | 132 | − |
| 258 | + | 128 | − |
| 260 | + | 220 | + |
| 677 | + | 208 | − |
| 329 | + | 177 | − |
| 380 | + | 187 | + |
| 546 | + | 172 | − |
| 309 | + | 96 | − |
| 309 | + | 163 | − |
| 341 | + | 209 | − |
| 300 | + | 158 | − |
| 928 | + | 169 | − |
| 261 | + | 96 | − |
| 1323 | + | 154 | − |
| 435 | + | 191* | + |
| 1401 | + | 153 | − |
| 752 | + | 121 | − |
| 342 | + | 136* | + |
| 546 | + | 234 | − |
| 426 | + | 210* | + |
| 1844 | + | 115* | + |
| 669 | + | 110 | − |
| 390 | + | 98 | − |
| 371* | | 223 | − |
| 1067 | + | 168* | + |
| 1022 | + | 160 | − |
| 286 | + | 92 | − |
| 528* | | 220 | − |
| 370 | + | 122 | − |
| | | 213* | + |
| | | 145 | − |
| | | 170 | − |
| | | 113 | − |
| | | 140 | − |
| | | 163 | − |
| | | 220* | + |

| | |
|---|---|
| % Agreement 82,4% | 93% |

*Discordant value.

These assays demonstrate the usefulness of the disposable visual sensor developed for detecting the presence of a high concentration of volatile sulphur compounds in breath (patients suffering from halitosis) and that he/she can be used by the own patients to control in situ, in an easy and quick way, the state of their illness.

1. A sensing element for halitosis, characterized in that said sensor is calorimetric and sensitive to volatile sulphur compounds.

2. A sensing element for halitosis, according to claim 1, which comprises a solid support and a redox colorimetric reagent sensitive to volatile sulphur compounds.

3. A sensing element for halitosis, according to claim 2, wherein said redox colorimetric reagent is 5,5′-dithiobis(2-nitrobenzoic acid).

4. A sensing element for halitosis, according to claim 2, wherein said redox calorimetric reagent is the metallic chelate Neocuproin-Cu(II).

5. A sensing element for halitosis, according to claim 2, wherein said support is a solid support of inorganic nature.

6. A device for the detection of halitosis, characterized in that it comprises: a sensing element for the halitosis according to anyone of claims 1 to 5 located inside a transparent tube.

7. A device for the detection of halitosis according to claim 6, which further comprises a mouthpiece coupled to one of the ends of said transparent tube and an instrument that regulates the sample volume that passes through said tube.

8. A sensing device for halitosis according to claim 6 or claim 7, which further comprises at least one sheet of a porous material to fix said sensing element in said transparent tube.

9. A sensing device for halitosis according to anyone of claims 6 to 8, which further comprises a white solid placed next to the sensing element.

10. A sensing device for halitosis according to anyone of claims 6 to 9 which experiences a visually perceptible colour change when the concentration of said volatile sulphur compounds in the gaseous sample to be analyses is higher than 200 ppb, preferably higher than 250 ppb.

11. A process for the detection of concentrations higher than a certain threshold of volatile sulphur compounds in gas samples, characterized in that it is based in a visually perceptible colour change.

12. A process according to claim 11, characterized in that said threshold is of 200 ppb, preferably 250 ppb.

13. A process according to claim 11 or claim 12, which comprises the following steps:
(1) passing a gas sample through an active sensing element;
(2) checking if, as a consequence of step 1, the sensing element has experienced a visually perceptible colour change.

14. A process according to anyone of claims 11 to 13, wherein the gas sample is breath from the buccal cavity of a patient.
